(19) United States
(12) Patent Application Publication
(10) Pub. No.: US 2024/0184241 A1
(43) Pub. Date: Jun. 6, 2024

(54) SYSTEMS AND METHODS FOR AN IMAGING DEVICE

(71) Applicant: RJS MEDIAGNOSTIX, New York, NY (US)

(72) Inventors: Balwant RAI, Rødovre (DK); Jasdeep KAUR, Rødovre (DK)

(21) Appl. No.: 18/561,257

(22) PCT Filed: May 26, 2022
(86) PCT No.: PCT/IB2022/054937

§ 371 Date: Nov. 15, 2023

Related U.S. Application Data
(60) Provisional application No. 63/194,522, filed on May 28, 2021.

(51) Int. Cl. G03H 1/04; A61B 1/00; A61B 5/00; G03H 1/00; G03H 3/00

(52) U.S. Cl. CPC G03H 1/0443; A61B 1/00009; A61B 5/0095; G03H 1/0005; G03H 3/00; G03H 2001/0033; G03H 2001/0445; G03H 2222/45; G03H 2223/12; G03H 2223/16; G03H 2223/24; G03H 2240/51

(57) ABSTRACT

Systems and methods for a camera system for imaging a diffuse medium such as mammalian tissue are provided herein. In one example, a camera system includes a light source configured to emit light, a first beam splitter positioned to split the emitted light into a reference beam and a transmission beam; an aperture though which the transmission beam traverses en route to an object, and where an object beam formed from light reflected off the object is configured to travel back through the aperture, a concave lens, a convex lens, a second beam splitter positioned intermediate the concave lens and the convex lens, and a detector. The detector is configured to receive at least a portion of the object beam and a portion of the reference beam to capture an image of an interference between the reference beam and the object beam.

### CROSS-REFERENCE TO RELATED APPLICATIONS

This application claims priority to U.S. Provisional Application No. 63/194,522, entitled “SYSTEMS AND METHODS FOR AN IMAGING DEVICE”, and filed on May 28, 2021. The entire contents of the above-identified application are hereby incorporated by reference for all purposes.

### TECHNICAL FIELD

This disclosure relates generally to diagnostic imaging, and in particular to the generation of three dimensional holographic medical images.

### BACKGROUND

Diagnostic imaging is an essential part of patient care. Medical images obtained during diagnostic imaging provide a mechanism for non-invasively viewing anatomical cross-sections of internal organs, tissues, bones and other anatomical areas of a patient, allowing a clinician to more effectively diagnosis, treat, and monitor patients. However, medical imaging is dominated by large, high-cost systems, making imaging impractical for many clinically useful tasks. Further, standard medical imaging machines require dedicated spaces and specially trained technicians, increasing medical costs and frequently leading to delays in treatment.

Mobile imaging increases the efficiency of healthcare services, provides greater accessibility, faster diagnoses, and in many cases decreases overall costs. Be that as it may, current systems suffer from issues with image resolution, weight, and connectivity. Further, as standard medical imaging devices are frequently limited to a single type of imaging, it is not unusual to need to schedule and undergo multiple types of imaging to obtain the necessary information for accurate diagnosis and monitoring. While multi-modal fusion of anatomical and functional information is an effective way to provide greater distinction between physiological and pathological conditions, if images are acquired using separate devices, the images require calibration and tracking to provide a common coordinate space. This is typically a complex and time consuming procedure prone to error as it may be challenging to match features taken using different modalities to create a combined image. Thus, there is a need for a portable, diagnostic tool that provides high fidelity multi-modal imaging for the diagnosis, monitoring, and treatment of disease.

### SUMMARY

Embodiments are disclosed herein for a camera system configured to provide high resolution multi-modal imaging. In one example, the camera system includes a light source configured to emit light in one or more wavelength ranges, a first beam splitter positioned to split the emitted light into a reference beam and a transmission beam, and an aperture though which the transmission beam traverses en route to an object. Light reflected off the object is configured to travel back through the aperture as an object beam. The camera system further includes a concave lens, a convex lens, a second beam splitter positioned intermediate the concave lens and the convex lens, and a detector configured to receive at least a portion of the object beam and a portion of the reference beam to capture an image of an interference between the reference beam and the object beam.

To the accomplishment of the foregoing and related ends, certain illustrative aspects of the system are described herein in connection with the following description and the attached drawings. The features, functions, and advantages that have been discussed can be achieved independently in various embodiments of the present disclosure or may be combined in yet other embodiments, further details of which can be seen with reference to the following description and drawings. The summary is provided to introduce a selection of concepts in a simplified form that are further described below in the Detailed Description. This summary is not intended to identify key features or essential features of any subject matter described herein.

### BRIEF DESCRIPTION OF THE DRAWINGS

FIG. 1 schematically shows a camera system according to an embodiment of the disclosure.

FIG. 2 is a flow chart illustrating a method for operating the camera system of FIG. 1, according to an embodiment of the disclosure.

FIG. 3 is a flow chart illustrating a method for generated a holographic image according to an embodiment of the disclosure.

FIG. 4 is a flow chart illustrating a method for generated phase-recovered holographic images according to an embodiment of the disclosure.

FIG. 5 is a graph showing mean image intensity as a function of exposure time for images obtained with a conventional holographic camera system and the camera system of FIG. 1.

FIG. 6 schematically shows a camera system according to another embodiment of the disclosure.

### DETAILED DESCRIPTION

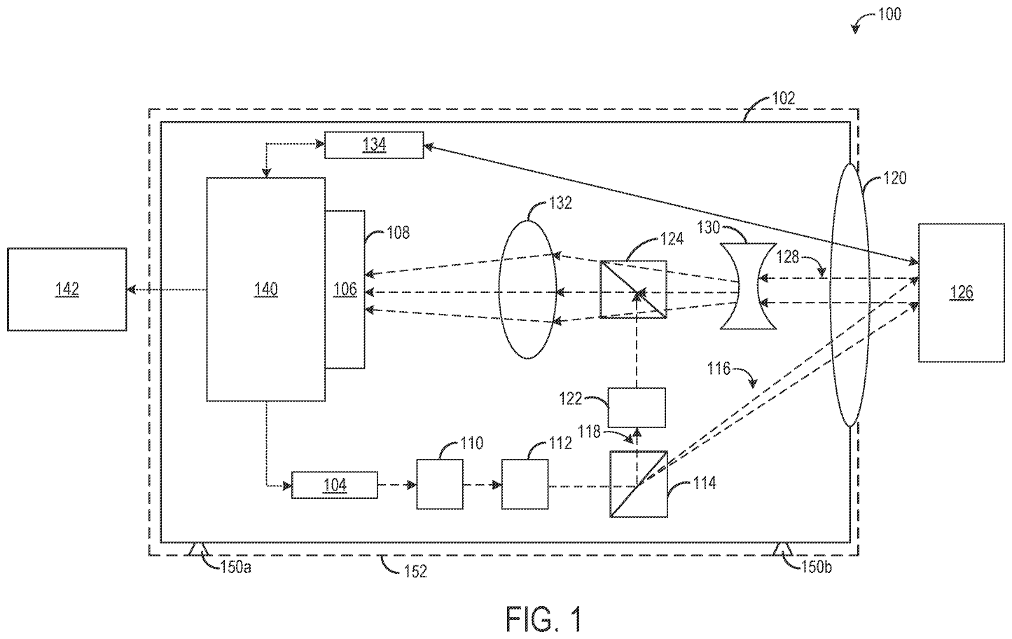

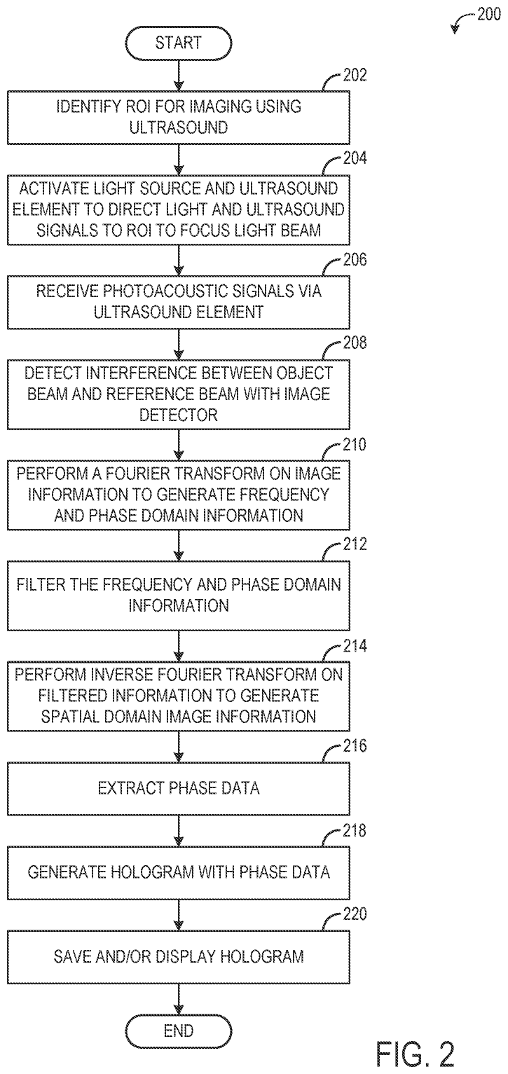

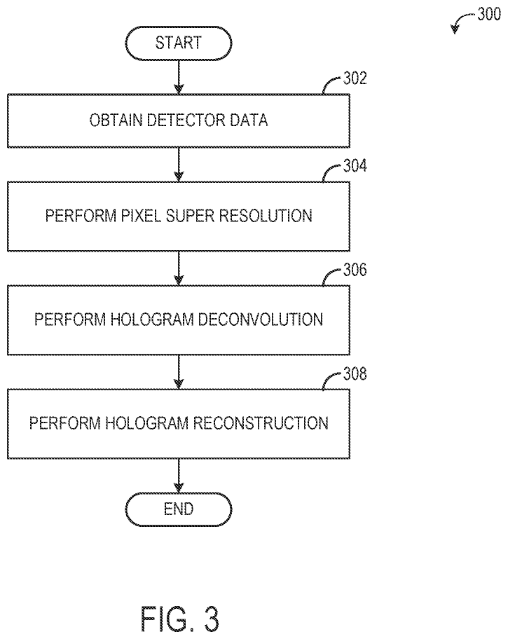

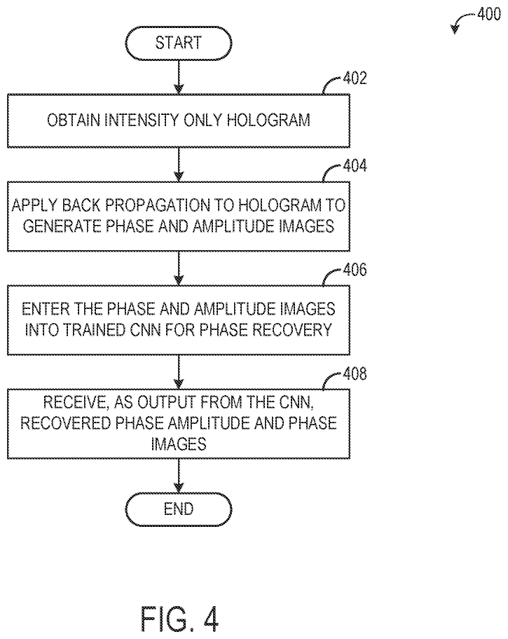

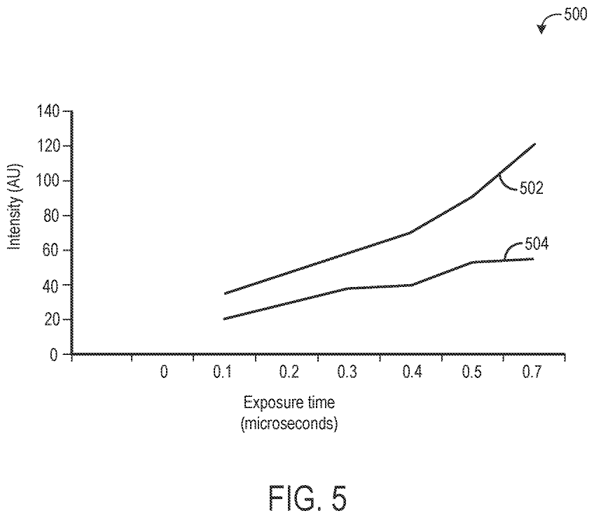

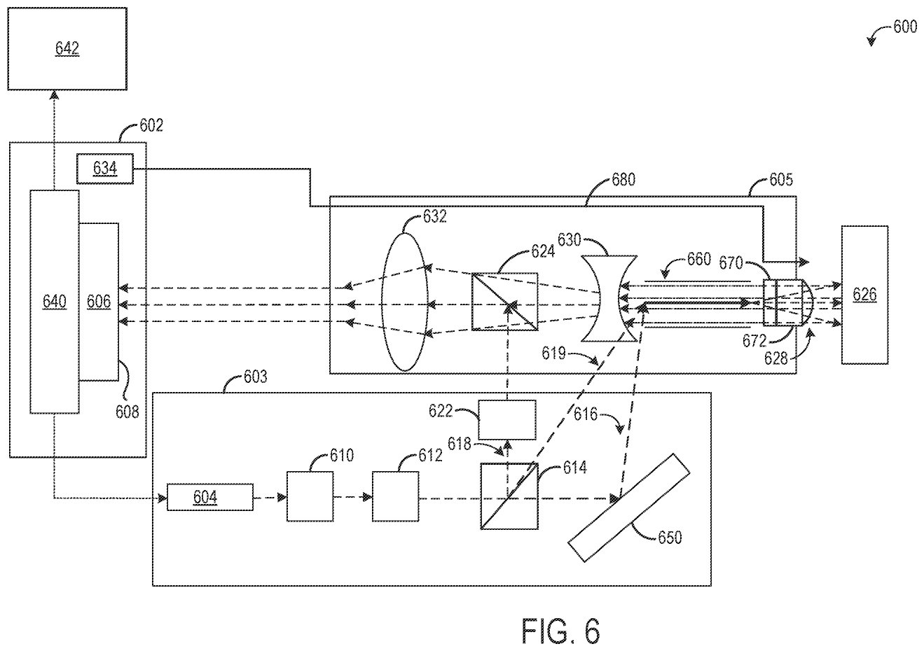

The present disclosure relates to a digital holographic system or device with a resolution of 2-10 μm and penetration depth of diffuse medium such as the human body of more than 100-200 mm. The digital holographic system described herein may also increase the field of view more than 16 degrees in comparison to standard modalities and uses multi-modal information and deep learning to create a hologram of the human body, allowing for three-dimensional viewing of systems and organs of interest. FIG. 1 schematically shows an example digital holographic system in the form of a camera system, which may be operated to obtain a holographic image according to the method of FIG. 2. The image information obtained by the camera system may be used to generate a holographic image according to the method of FIG. 3 and/or according to the method of FIG. 4. The camera system may include a combination of a concave and a convex lens, which may increase the field of view of the camera and reduce the exposure time, as shown by the graph of FIG. 5. FIG. 6 schematically shows an example system in the form of an endoscope, which may be operated to obtain a holographic image according to the methods of FIGS. 2, 3, and/or 4.

FIG. 1 shows a camera system 100 according to an embodiment of the disclosure. The camera system 100 includes a housing 102 that houses a plurality of components including a light source 104 and a detector 106 having a detector plane 108. The light source 104 includes a source of coherent light, such as one or more lasers. The lasers may emit light in the mid-infrared or infrared range (e.g., in a range of 780 nm-1 mm, in a range of 720-1700 nm, or in a range of 800-1700 nm) with a width of 4 to 6 ns (laser fluence). In some aspects, the light source may be controlled to emit light at a pulse repetition rate of 10 Hz. While any type of useful light may be used, in some examples, the light source may be controlled to emit light at one or more wavelengths and/or wavelength ranges, for example, ultraviolet (UV) light (e.g., 180-400 nm, preferably 300-400 nm), alternative light at a wavelength of 350 nm, and/or visible light of 400-700 nm. The wavelength(s) of the light source may be selected based on the object being imaged and/or a diagnostic goal of the imaging, as different wavelengths may penetrate tissue to different depths and different wavelengths may provide contrast for visualizing different types of tissue or cells. For example, to differentiate oxygenated blood from deoxygenated blood, two different wavelengths of light (e.g., 720 nm and 820 nm) may be emitted in an alternating fashion.

The camera system 100 further includes a prism 110 and a diffuser 112. The prism 110 may be a right-angle prism positioned to reflect the incident laser beam from the light source to the diffuser 112. The diffuser 112 is configured to homogenize the light beam. The diffuser 112 may have a roughness of 1.5-2.3 μm, a depth 10 to 30 μm, and a diffusion angle of 20 to 70°. The camera system 100 further includes a first beam splitter 114 downstream (in a light-traveling direction) from the diffuser 112. The first beam splitter 114 may split the incident beam of light (after traveling through the prism and diffuser) into a first beam 116 and a second, reference beam 118. The first beam 116 may travel out of the housing 102 via an aperture 120. The reference beam 118 may be maintained in the housing 102, and may travel to a neutral density filter 122 and then a second beam splitter 124. The neutral density filter 122 may optimize the intensity of the reference beam. In some examples, the neutral density filter 122 may have properties that are based on the wavelength(s) of light being emitted, e.g., a neutral density filter of 40 μm may be used when the light source is configured or controlled to emit infrared light. Each neutral density filter in the series, ranging from ND-0.3 to ND-70 (Olympus, USA), has an incrementally lower extinction coefficient. This filter set jointly gives a uniform series of filters for adjusting illumination intensity. The neutral density filter 122 may thereby include one or more filters in the series of filters described above (e.g., ND-0.3 to ND-70). An ND-70 filter transmits (or passes) 70 percent of the incident light from the light source, an ND-0.3 filter transmits 0.3 percent of incident light, etc.

The first beam 116 may impinge on an object 126, causing some of the first beam 116 to reflect off the object 126. At least a portion of the reflected light may travel back into the housing 102 via the aperture 120, forming an object beam 128. The object beam may travel through a first lens 130. The first lens 130 may be a concave lens with suitable parameters, such as D 25 mm, f 50 mm or other suitable parameters, that may cause the light to diverge (e.g., thereby creating a demagnified virtual image of the object).

The camera system 100 further includes a second lens 132 positioned between the first lens 130 and the detector 106, with the second beam splitter 124 positioned between the first lens 130 and the second lens 132. The second beam splitter 124 may be a cube beam splitter that combines the object and reference beams, such that the reference beam travels to the detector 106 off-axis from the object beam. The second lens 132 may be a convex lens (D 25 mm, f 50 or 80 mm or other suitable parameters) which is used to converge the light (e.g., from the second beam splitter 124) and transmit the image ahead of the detector plane 108. The combination of convex and concave lenses increases the number of photons directed to the detector 106 and reduces exposure time. For example, because the convex lens converts the light (e.g., converges it) the convex lens can change the effective/image size of the object. Converging the light changes the intensity of the photons. When the intensity increases, the time of exposure can be decreased and the light is directed toward the detector to capture the maximum wide angle image. Thus, the combination of the concave and convex lenses widens the angle, increases the resolution, and reduces the exposure time. By positioning the beam splitter intermediate the first and second lenses, the widened angle may be achieved by the first lens 130 without disrupting the interference between the object beam and the reference beam that is captured by the detector to create the hologram, as explained in more detail below.

When the object beam and the reference beam are combined via the second beam splitter 124, their light waves intersect and interfere with each other, creating an interference pattern that is detected by the detector 106. The detector 106 may be a digital hologram charge-coupled device (CCD) (3456×2892 pixels, pixel size=1.2-2.1 μm, monochrome) or a complementary metal-oxide-semiconductor (CMOS) image sensor (e.g., with a pixel size of 1.1 μm).

The first (e.g., concave) lens 130 creates a demagnified virtual image of the object which reduces the spatial frequency detected by the detector 106. Thus, the camera system 100 may be used to record large size objects at shorter propagation distances than in traditional digital holography. As the concave lens diverges the object light beam, fewer photons are directed to the detector (in the absence of the convex lens). The second (e.g., convex) lens 132 takes the light diverged from the concave lens and converges the light toward the detector 106, thereby increasing the number of photons directed to the detector to compensate for the dispersion from the concave lens. As will be explained in more detail below, the interference pattern detected by the detector 106 may be used to generate a holographic image. The combination of lenses described herein, when used to generate holographic images, results in higher average intensities of reconstructed images at lower exposure time as compared to systems that include a concave lens without a convex lens. Further, the field of view of the camera system 100 may be wider (e.g., 25 degrees) compared to conventional holographic cameras (e.g., 5 degrees).

In some embodiments, a linear polarizer or a circular polarizer may be included in the camera system 100 to polarize shifted signal to have the same polarization orientation as light source reference wavefront. The polarizer may be positioned parallel to the diffuser or in front of the detector.

In some examples, the camera system 100 further includes an ultrasound element 134. The ultrasound element 134 may include one or more transducer elements, e.g., an array of transducer elements, such as a linear 128-element transducer array. The ultrasound element 134 may be controlled in a transmit mode to transmit ultrasound signals that may be used to modulate the light of the object beam. For example, the ultrasound waves and light waves from the object beam may meet at the same time at a specific area of interest (e.g., on the object). The ultrasound wave may cause a shift in the wavelength of the object beam (change in phase as well as amplitude). This process, referred to as optical phase conjugation, focuses light inside scattering media (e.g., the object) by first measuring and then phase conjugating (time reversing) the scattered light field emitted from a guide star which is positioned at a targeted focusing location deep inside a scattering medium. Focused ultrasound is provided to noninvasively provide a (virtual) guide star, which is freely addressable within tissue. Due to the acousto-optic effect, a portion of the light passing through the ultrasonic focus changes its frequency by an amount equal to the ultrasonic frequency. These “ultrasound-tagged photons” emitted from the virtual guide star are then scattered as they propagate through a turbid medium such as the body toward the detector.

While any ultrasound transducer may be used, in some examples, the ultrasound element may include ultrasound transducers comprised of polyimide thin films coated on a silicon wafer with thickness 5-20 μm. A second polyimide layer be positioned on the polyimide thin films (with an intervening patterned layer of gold) with holes etched therein. The second polyimide layer may have a thickness is 50-120 μm. PZT in the holes may be filled with silver paste. The transducer elements include top electrodes of gold or other suitable electrode and are wire-bonding with copper or other suitable materials. Uniform imaging region with high radial resolution is 5-100 μm and the tangential resolution is 10-400 μm formed by the combination of combined foci of elements.

In some aspects, the ultrasound element 134 may be configured and controlled in such a way to change the frequency and focus of a specific area. Ultrasonic transducers can scan out of 70+/−degree volume and are focused with good spatial resolution since most tissue components have similar acoustic impedance. The emitted light (e.g., emitted from the light source as described above) that is localized at the focal region of the ultrasound wave can be diffracted and also frequency shifted by the ultrasound. This frequency shifted light propagates out of the tissue and a fraction of this light can be captured by a light detector (e.g., the detector 106). Time-reversed photoacoustic wave guided time-reversed ultrasonically encoded (TT) optical focusing incorporates ultrasonic focus guided via time reversing photoacoustic signals, and the ultrasonic modulation of diffused coherent light with optical phase conjugation to get active focusing of light into a scattering and diffused medium. In other words, the ultrasound element 134 may be used to focus the light source in the tissue.

In some examples, the ultrasound element 134 may be controlled in a receive mode to receive photoacoustic signals generated by the object due to thermal expansion resulting from the object beam. For example, when the object is human or animal tissue, absorption contrasts within the tissue are acoustically detected via the photoacoustic effect in which initial acoustic pressure arises if chromophores undergo a heat increase after absorbing the incident light (e.g., laser) energy. By selecting the light wavelength, specific absorption agents can be identified due to their different absorption coefficients, e.g., deoxyhemoglobin is more sensitive at 720 nm while oxyhemoglobin is more sensitive at 820 nm. The infrared laser pulses are delivered into diffused media (e.g., the tissue) and part of the energy will be absorbed and converted into heat, leading to thermal expansion. The ultrasound element 134 receives these photoacoustic signals.

Based on the signals received at the detector 106 and/or ultrasound element 134, a three-dimensional image is formed of the object. The image may represent a combination of optical, ultrasonic, and photoacoustic signals as one image due to one detector that is able to detect the optical, ultrasonic, and photoacoustic information. For example, the detector 106 may be a resonant IR sensor, such as an aluminum nitride piezoelectric nano-plate resonant detector comprised of a silicon wafer, a platinum inter-digital transducer, an aluminum nitride thin film (which acts as a resonator), and a top layer of Si₃N₄ (which acts as an IR absorber). In some examples, the sensor may include a plasmonic absorber on the aluminum nitride thin film, such as a layer of gold. The nano-plate may be coupled to a CMOS readout circuit. The detector 106 may be configured as 2000 pixels by 1000 pixels with pixel pitch of 1.2-7.9 microns. However, in some examples, a separate detector or chip or sensor might be used to detect the ultrasonic and photoacoustic information (e.g., the ultrasound element 134). The aluminum nitride thin film may have lower piezoelectric coefficients and low relative permittivity, which results in piezoelectric micromachined ultrasonic transducers with lower pressure sensitivity in transmitting and lower charge output in receiving (e.g., than conventional ultrasound transducers). Therefore, aluminum nitride piezoelectric micromachined ultrasonic transducers make ultrasound pulse-echo detection more challenging and as such a low-noise and impedance matched local pre-amplifier may be utilized. However, the aluminum nitride piezoelectric micromachined ultrasonic transducers may exhibit increased sensitivity to detect different waves of different frequency and/or energy.

In some aspects, multi-modal device with both the ultrasound emitter and pulsed laser infrared optical imaging with wavelength of 720-1700 nm (such as 800-1700 nm), ultraviolet (UV 180-400 nm, 300-400 nm, or 350 nm), or/and visible light with 400-700 nm automatically transmits the laser and ultrasound so that they meet at the same time and at the same location. Each image pixel of the co-registered information is detected with a sensitive detector to obtain images using only one laser pulse per pixel i.e., display pixel which might be liquid crystal or stretchable crystal for modulating the different parameters of the image information light such as phase and intensity for construction of a three-dimensional holographic image. In some examples, the light emitted by the light source may be fluorescent light.

The camera system 100 may further include anti-vibration rubber mounts, such as mounts 150a and 150b, to minimize the effect of ground, hand, or tripod vibrations. The mounts 150a, 150b may be coupled to the housing 102. In some examples, the housing 102 may be an inner housing and the complete system is housed in an outer housing 152 to diminish the effect of environmental vibrations on the functioning of the camera. In such examples, the mounts 150a, 150b may be coupled between the inner housing 102 and the outer housing 152. While two mounts are shown in FIG. 1, it is to be understood that any number of mounts may be included without departing from the scope of this disclosure. The outer housing 152 may be configured to be coupled to a tripod, held by a hand of a user, or positioned/stabilized via another suitable manner. In some embodiments, two off-axis illumination directions of the rotating the object are used to increase the field of view and reduce the exposure time as well as reduce the effect of vibration.

The camera system 100 further includes a controller 140. The controller 140 may be configured to control the light source 104 (e.g., control the pulse frequency) and the ultrasound element 134. Further, the controller 140 may be configured to receive ultrasound and/or photoacoustic information from the ultrasound element 134 and receive optical information (e.g., the interference pattern) from the detector 106. The controller 140 may be configured to generate one or more images based on the ultrasound, photoacoustic, and/or optical information, or the controller 140 may be configured to send the ultrasound, photoacoustic, and/or optical information to an external computing device 142 for processing.

For example, the controller 140 may include a memory and one or more processors. The processor may control the light source, ultrasound element, and/or detector to acquire the image information described herein according to instructions stored on the memory of the controller. The processor may be in electronic communication with a display device and/or the external computing device 142, and the processor may process the image information into images for display on the display device. The processor may include a central processing unit (CPU), according to an embodiment. According to other embodiments, the processor may include other electronic components capable of carrying out processing functions, such as a digital signal processor, a field-programmable gate array (FPGA), or a graphic board. According to additional embodiments, the processor may include multiple electronic components capable of carrying out processing functions. For example, the processor may include two or more electronic components selected from a list of electronic components including: a central processor, a digital signal processor, a field-programmable gate array, and a graphic board. The memory may comprise any known data storage medium.

The camera system 100 may also include motion and temperature sensors, in some examples. The output from the motion sensor may be used to monitor the vibration of the camera system 100, which might affect the quality of the three-dimensional images generated by the camera system 100. Thus, if motion is high, obtained image information may be discarded, motion correction techniques may be applied to any generated images, and/or a notification may be output to an operator. The output of the temperature sensor may be used to monitor the temperature of the tissue being imaged. If the temperature increases above a threshold, then the camera system 100 may be automatically deactivated.

Thus, the camera system 100 may be used to obtain image information of an object, such as tissue of a human subject, and the image information may be used to generate one or more images, which may be three-dimensional images (e.g., holograms). The image information is detected with the detector 106 while the reference beam is illuminating the detector and the three-dimensional image takes an interference between the reference beam and the incoming object beam signal. Frequency and phase domain image information (e.g., optical, ultrasound, and/or photoacoustic image or information) may be generated by performing a Fourier transform operation on the optical, ultrasound, photoacoustic image or information detected at the detector and/or ultrasound element. Filtered frequency domain image information may be generated by applying a specific filter to the frequency domain image to isolate a frequency representing the interference between the reference beam and the incoming image or information signal (e.g., the object beam). Spatial domain image information is generated by performing an inverse Fourier transform. Phase data is extracted from the spatial domain image information generated by performing an inverse Fourier transform. In some examples, all optical, ultrasonic, and/or photoacoustic information is combined to get single information or image by using generative adversarial network and a residual network.

In some embodiments, the camera system is detecting not only photoacoustic waves but also optical signals and ultrasonic signals to create three-dimensional images. In some embodiments, the system is detecting not only photoacoustic waves but also optical signals to create three-dimensional information of the object. In some embodiments, the system is detecting not only optical signals but also ultrasonic signals to create three-dimensional information of the object. In some embodiments, the system is detecting photoacoustic signals only.

In some embodiments, the system is configured to perform photoacoustic wavefront shaping, with high or 10 times higher signal to noise ratio, multipoint focusing with lower pulse repetition rates, increasing the speed of scanning by the combination of wavelet denoising and correlation detection or other suitable methods.

In an embodiment, the SNR is improved by integrating a low frequency transducer (PVDF) on top of a low frequency piezoelectric element (PZT), such as the aluminum nitride piezoelectric micromachined ultrasonic transducers described above.

As explained above, in some examples, aluminum nitride piezoelectric technology based nano-Electro-Mechanical Systems detector (e.g., the nano-plate IR detector described above) for piezoelectric ultrasonic, infrared detector and UV or optical and/or multi-spectral imaging arrays based on a plasmonic piezoelectric material with high resolution, high SNR, ultra-fast response systems are manufactured using MEMS technology. The combination of aluminum nitride piezoelectric technology and the plasmonic based technology improves the resolution of IR, UV, and photoacoustic imaging systems.

In some embodiments, a MEMS spatial light modulator may be included in the camera system 100 (e.g., in front of the light source) to increase the zone of viewing and the zone of the angle of IR, UV, and photoacoustic imaging systems.

Turning now to FIG. 2, a method 200 for operating a camera system to acquire digital holographic images is shown. Method 200 is described with regard to the systems and components of FIG. 1, though it should be appreciated that the method 200 may be implemented with other systems and components without departing from the scope of the present disclosure. Method 200 may be carried out according to instructions stored in non-transitory memory of a controller of a camera system, such as controller 140 of FIG. 1. In some examples, one or more aspects of method 200 may be performed on a computing device in communication with the camera system, such as computing device 142 of FIG. 1.

At 202, method 200 optionally includes identifying a region of interest (ROI) for imaging using ultrasound. For example, a user of the camera system may position the camera system to image an object, such as human tissue. The user may enter a user input (e.g., directly on the camera system or via a coupled computing device) requesting the camera system acquire ultrasound images. In response, the ultrasound element of the camera system (e.g., ultrasound element 134) may be activated to transmit ultrasound signals to the object and receive ultrasound echoes from the object. The controller of the camera system and/or the external computing device may process the received echoes to generate one or more ultrasound images that are output for display on a display device. The user may then reposition the camera system until the desired ROI is within the field of view of the camera system.

At 204, the light source of the camera system (e.g., light source 104 of FIG. 1) is activated in order to emit light to the ROI of the object. The light source may be activated to emit light in one or more desired wavelengths, depending on the imaging protocol or diagnostic goal of the imaging. The light source may be pulsed at a suitable pulse rate, such as a pulse repetition rate of 10 Hz. Further, when more than one wavelength of light is emitted, the light source may be controlled to emit the different wavelengths of light in an alternating manner or the light source may be controlled to emit light of a first wavelength for a first duration (e.g., to obtain image information sufficient for generating a first image) and then emit light of a second, different wavelength for a second duration (e.g., to obtain image information sufficient for generating a second image).

At the same time that the light source is activated to emit the light to the object, the ultrasound element may also be activated to transmit ultrasound waves to the ROI. The ultrasound waves may focus the light beam. As explained above, due to the acousto-optic effect, a portion of the light passing through the ultrasonic focus changes its frequency by an amount equal to the ultrasonic frequency in order to generate “ultrasound-tagged photons.” The ultrasound element may be activated with transmit parameters that are the same or different as when the ultrasound element was activated to generate the ultrasound images. For example, the ultrasound element may be activated in the transmit mode with a frequency of 1.3 MHz for a radius of 110 micrometers and 1.90 MHz for an area of 0.04 m².

At 206, photoacoustic signals are received via the ultrasound element. For example, the emitted light, when it impinges on the object, may cause thermoelastic expansion of the object as part of the light is absorbed by the object and hence generates heat. The thermoelastic expansion results in the emission of ultrasonic signals that can be detected with the ultrasound element or another suitable detector. In some examples, the light source may be emitted before the ultrasound element is activated and the photoacoustic signals may be obtained upon the light source reaching the object. The photoacoustic signals may then be used to determine the ultrasound transmit parameters for focusing the light beam at the object. This control of the light source and the ultrasound element to generate the ultrasound-tagged photons may be performed in a manner similar to that described in Zhang, Juze et al. “Time-reversed photoacoustic guided time-reversed ultrasonically encoded optical focusing.” arXiv: Optics (2020), which is incorporated herein by reference in its entirety. Further, the photoacoustic signals may be used to generate an image, whether alone or combination with the optical signals and/or ultrasound signals.

At 208, the interference between the object beam and the reference beam is detected with the image detector (e.g., detector 106 of FIG. 1). As explained above, the camera system includes a first beam splitter to split the light from the light source into a beam that is directed to the object and a beam that is directed back to the detector, referred to as the reference beam. The light that reflects off the object and returns to the camera system is referred to as an object beam. The object beam and the reference beam may be brought together ahead of the detector (e.g., via a second beam splitter), and the reference beam may interfere with the object beam. This interference is detected by the image detector (which is also referred to as the optical detector herein). The output of the detector (e.g., the detected interference) may be referred to as image information, as the information is usable to generate an image, as described below. In some examples, the optical (e.g., interference information), ultrasonic, and/or photoacoustic information are combined to get a single set of image information by using a generative adversarial network and a residual network. The generative adversarial network (GAN) may include a generator and a discriminator. In some aspects, the generator aims to generate a fused image with major optical (e.g., infrared), ultrasonic, and/or photoacoustic information or other intensities together with additional visible, infrared, ultrasonic, and/or photoacoustic gradients, and the discriminator forces the fused image to have more details in visible images information. Furthermore, the GAN also allows for the fusion of image information with different resolutions. For example, the GAN may be trained with training data that includes a plurality of concatenated sets of images, where each concatenated set of images includes an optical image (e.g., a hologram as obtained by the CCD or CMOS detector of the camera), an ultrasound image (e.g., generated from ultrasound echoes received from the ultrasound element of the camera), and/or a photoacoustic image of the same imaging subject and region of interest. Each concatenated set of images may be entered into the generator, which may output a fused image based on that concatenated set of images. The fused image may be entered into the discriminator along with a selected image of the concatenated set of images, such as the optical image. The GAN may then establish an adversarial game between the generator and the discriminator, which will result in increasing amounts of detail from the selected image being included in the fused image. Once the generator generates fused images that cannot be distinguished by the discriminator (e.g., the discriminator thinks the images are only the selected images, e.g., the optical images), the GAN may be determined to be trained. Once trained and validated, the optical (e.g., interference information), ultrasonic, and/or photoacoustic information obtained with the camera system as described herein may be concatenated and entered as input to the trained generator, which may output a final fused image.

At 210, a Fourier transform may be performed on the image information in order to generate frequency and phase domain information. At 212, the frequency and phase domain information may be filtered to generate filtered frequency and phase domain information. The filtering may include applying a specific filter to the frequency domain information to isolate a frequency representing the interference between the reference beam and the object beam. The filter may be applied via an adaptive filtering process based on iterative thresholding and region-based selection. This combination gradually selects the optimal frequency component boundary and uses shape recognition to find the optimal frequency component for different holograms. Phase shift is performed in the spatial frequency on two symmetrical areas in the frequency domain after transform of the hologram. Frequency analysis is performed to get proper reconstruction. Process or method of iterative thresholding and region-based selection is done by applying the global threshold level (Xue L, Lai J, Wang S, Li Z. Single-shot slightly-off-axis interferometry-based Hilbert phase microscopy of red blood cells. Biomed Opt Express. 2011; 2(4):987-995. Published 2011 Mar. 29. doi:10.1364/BOE.2.000987, incorporated by reference herein in its entirety) to the intensity of fast Fourier transform hologram to lead to the binary image information followed by applying region recognition process of the same via using regionprops MATLAB. The global threshold level may be repeated after increasing the threshold level about 1 to 2% in the first step and increasing the threshold levels may be repeated until the minimum number of regions reaches three or four. Binary image information and the regionprops function from the above-mentioned step may be used to choose a proper frequency component boundary and used as a filtering window and box boundary data. A Gaussian function is performed to smooth the edge of final filtering window. In some aspects, this process may be automatic. The shapes and sizes of these two symmetrical areas can vary according to different imaging conditions.

At 214, an inverse Fourier transform is performed to convert the filtered information to the spatial domain, thereby generating spatial domain information (e.g., frequency and phase spatial domain information). At 216, the phase data is extracted from the spatial domain information. The phase data is extracted from the spatial domain information performing another inverse Fourier transform. At 218, a hologram is generated with the phase data. At 220, the hologram (which may also be referred to as a holographic image) is saved in memory and/or displayed on a display device. Method 200 then ends.

Thus, focused ultrasound may be provided to guide a voxel on the area of interest and be followed by focusing the light source, such as infrared light, in the area of interest labeled by the voxel so that the ultrasound and emitted light meet at the same time at the specific area of interest at the voxel. The shift wavelength of the laser with the ultrasound wave (change in phase as well as amplitude) is detected by very sensitive fast image pixel arrays resulting in the construction of the three-dimensional image. At the same time, an ultrasonic three-dimensional image may be constructed. Absorption contrasts within the tissue may be acoustically detected via the photoacoustic effect in which initial acoustic pressure arises if chromophores undergo a heat increase after absorbing the incident light energy. By selecting the light wavelength that is emitted by the light source, specific absorption agents can be identified due to their different absorption coefficients, e.g. deoxyhemoglobin is more sensitive at 720 nm while oxyhemoglobin is more sensitive at 820 nm. A list of possible wavelengths, pulse times, and uses is shown in Table 1 below.

TABLE 1

| | Wavelength | Time | Uses |
|---|---|---|---|
| 1 | 810, 940, 1200 nm, | 50-200 ns | Surgery |
| | 1470 nm | | |
| 2 | 650-900 nm | 10 to 100 ns | Head and neck cancer |
| 3 | 700-850 nm | Less than 10 | oxy-, deoxy-, and total- |
| | | microseconds | hemoglobin |
| | | | concentrations |
| 4 | 700-850 nm, 1064-nm | Less than 20 | Prostate cancer |
| | | microseconds | |
| 5 | 720 nm-820 nm, | 10-ns | Breast cancer |
| | 1200 nm | | |
| 6 | 350-450 nm, 720-850 nm | Less than 10 | Oral cavity precancerous |
| | | microseconds | and cancer |
| 7 | 350-450 nm, 700 to 970 | 5 to 10 seconds | Skin precancerous and |
| | nm | | cancer as well as other |
| | | | body part cancer |

In some examples, as explained above, the photoacoustic signals may be used to generate an image. For example, a three-dimensional back projection method may be used to reconstruct a three-dimensional structure without any motion artifacts from the three-dimensional information (e.g., photoacoustic information). Raw data, after complete data acquisition, are reconstructed as an image, based on a newly designed algorithm. Joint reconstruction method is applied to avoid an error (Q. Sheng et al., “Photoacoustic computed tomography without accurate ultrasonic transducer responses,” Proc. SPIE, 9323 932313 (2015), incorporated by reference herein in its entirety). Three dimensional images are formed.

FIG. 3 is a flow chart illustrating a method 300 for generating a hologram. In some examples, method 300 may be performed as an alternative to the hologram generation described above with respect to FIG. 2, e.g., the optical, ultrasonic, and/or photoacoustic information obtained as described above with respect to FIG. 2 may be used according to the method of FIG. 3 to generate a hologram. In other examples, the hologram that is generated according to FIG. 2 may be generated using the method 300.

At 302, detector data is obtained. The detector data may include raw data (e.g., unprocessed) from detector 106 and/or ultrasound element 134, in some examples. In other examples, the detector data may include processed detector data, e.g., the filtered spatial domain information described above with respect to FIG. 2. At 304, a pixel super resolution process is performed on the detector data. At 306, a hologram deconvolution is performed. At 308, a hologram reconstruction is performed to generate a hologram. In some examples, the hologram that is generated via the hologram reconstruction may lack phase information (e.g., the hologram may be an intensity-only hologram), and thus in some examples a phase recovery may be performed in order to generate amplitude and phase images. An example phase recovery process is described below with respect to FIG. 4. Method 300 then ends.

The pixel super-resolution process may be applied to mitigate resolution loss. Pixel super-resolution is applied which is based on wavelength scanning (Luo, W., Zhang, Y., Feizi, A. et al. Pixel super-resolution using wavelength scanning. Light Sci Appl 5, e16060 (2016), incorporated by reference herein in its entirety). Other methods of pixel super resolution such as the sensor array or the sample shifting the illumination source might be used. The object is refinement of this initial pixel function, deconvolution of the hologram of an object via using a blind deconvolution algorithm, i.e., a built-in MATLAB routine providing maximum likelihood estimation for both the pixel function and the unblurred image. After 20 to 40 iterations of blind deconvolution algorithm a refined pixel function was obtained for the detector. By evaluating reconstructed images, one can measure the effect of the estimated pixel function, and the pixel function with the best performance can be treated as an approximation to the real pixel function. The objects reconstructed from the deconvolved holograms are assessed via measuring the modulation depths or/and the width of averaged cross-section profiles. Combining of all these evaluation results from object and used this combination as the ‘cost function’ for pixel function optimization.

FIG. 4 is a flow chart illustrating a method 400 for applying a phase recovery process to a hologram in order to generate phase-recovered phase and amplitude images from the hologram, which may be of higher quality than non-phase recovered images.

At 402, an intensity-only hologram is obtained. The intensity-only hologram may be generated according to the methods of FIGS. 2 and/or 3. At 404, a back-propagation is applied to the hologram in order to generate phase and amplitude images. The phase and amplitude images that are generated via the back-propagation may lack phase information, which may result in image artifacts and suppression of image information, as explained below. At 406, the phase and amplitude images are entered as input to a trained network (such as a convolutional neural network (CNN)). The trained network/CNN is trained to perform phase recovery on the images and reconstruct phase-recovered images. At 408, recovered-phase amplitude and phase images are received as output from the trained network/CNN. These images may be saved and/or output for display on a display device. Method 400 then ends.

In one embodiment, a deep neural network is used for image reconstruction and phase recovery as well as analysis of the holographic image, as explained in Rivenson, Y., Zhang, Y., Gunaydin, H. et al. Phase recovery and holographic image reconstruction using deep learning in neural networks. Light Sci Appl 7, 17141 (2018), which is incorporated herein by reference.

Therein, using trained model non-iterative image reconstruction, twin-image suppression and phase recovery is performed. Images recovered by the model are comparable to those obtained via multi-height reconstruction method by using only a single back-propagated hologram. Firstly, deep learning-based phase recovery and holographic image reconstruction framework involves training of the neural network in which learning the statistical transformation between a complex valued image from the back-propagation of a single hologram intensity of the object. The same object's image is reconstructed using a multi-height phase recovery algorithm which acts as a gold standard for the training phase by using at least 6 to 10 hologram intensities acquired at different sample-to-sensor distances. A simple back-propagation of the hologram, without phase retrieval, results in severe twin-image and self-interference related artifacts, hiding the phase and amplitude information of the object. This one-time training/learning process leads to a fixed deep neural network which is used to blindly reconstruct, using a single hologram intensity, phase and amplitude images of any object, free from twin-image and other undesired interference related artifacts.

In one embodiment, novel convolutional neural networks (CNNs) may include a deep CNN operating on the t space or amplitude, a deep CNN operating on an image domain (ICNN), and interleaved data consistency operations. Each CNN is trained to minimize the loss between the reconstructed and corresponding fully sampled. This method is improved SNR, restoring tissue structures and removing aliasing artifacts. The Skip connections are used as extra connections between nodes in different layers of a neural network to facilitate denoising ability. Training is done via an incremental manner. Separate training of each CNN may be performed i.e., only one last network is trained while the previously trained networks are fixed.

FIG. 5 shows a graph 500 plotting mean image intensity as a function of exposure time for the camera system of FIG. 1 and a conventional holographic camera. As explained above with respect to FIG. 1, the camera system disclosed herein includes a combination of a concave and convex lens. In contrast, conventional holographic cameras may only include a single concave lens (and may lack a convex lens). As shown by FIG. 5, the combination of the concave and convex lenses results in increased mean image intensity as exposure time increases (shown by line 502), relative to a system including only a concave lens (shown by line 504). As a result, exposure time may be reduced without compromising image quality.

FIG. 6 shows a camera system 600 according to another embodiment of the disclosure. The camera system 600 may be configured for generating three-dimensional holographic images and may be configured for use in an endoscope, at least in some examples. Aspects of camera system 600 are the same as or similar to aspects of camera system 100, and the description of components of camera system 100 that are the same as or similar to components of camera system 600 apply herein. Camera system 600 includes a detector 606 having a detector plane 608, a controller 640, and an ultrasound element 634. The detector 606, the controller 640, and the ultrasound element 634 may be the same as detector 106, controller 140, and ultrasound element 134, respectively, of FIG. 1. The camera system 600 further includes a light source 604, a prism 610, a diffuser 612, and a first beam splitter 614 that are the same as the light source 104, the prism 110, the diffuser 112, and the first beam splitter 114, respectively, of FIG. 1. Thus, light from the light source 604, after passing through prism 610 and diffuser 612, is split by first beam splitter 614 into a transmission beam 616 and a first reference beam 618. The first reference beam 618 may travel to a neutral density filter 622 and then a second beam splitter 624. The neutral density filter 622 may be the same as neutral density filter 122.

The camera system 600 further includes a spatial light modulator 650 positioned in a path of the transmission beam 616. The transmission beam 616 is configured to impinge on the spatial light modulator 650 and eventually be directed to an object 626. The spatial light modulator 650 may comprise, but is not limited to, a magneto-optic, liquid crystal, deformable mirror, multiple quantum well, acoustic-optic Bragg cells, liquid crystal on silicon, and/or computer-based spatial light modulator. The spatial light modulator 650 may modulate the transmission beam 616, e.g., phase shift the transmission beam 616. In some examples, the spatial light modulator 650 may have a resolution of 1542×1020 pixels and a pixel pitch of 10 μm.

The transmission beam 616 may travel to a partially-reflective mirror 670. The partially-reflective mirror 670 may be comprised of a 12 mm thick layer of titanium on 0.85 mm glass slide, at least in some examples, which allows low-coherence full-field phase-shifting holography to facilitate imaging of live samples. In some examples, a second reference beam 619 may be directed from the first beam splitter 614 to the partially-reflective mirror 670 along with the transmission beam 616. The second reference beam 619 may be time-delayed relative to the transmission beam 616 and may have a different phase than the transmission beam 616 (due to the modulation of the transmission beam 616 by the spatial light modulator 650). The partially-reflective mirror 670 reflects the second reference beam 619. The transmission beam 616 travels through the partially-reflective mirror 670 and optionally through a lens system 672 before impinging on the object 626. Light reflecting off the object 626 travels back into the camera system 600 to thereby form an object beam 628. Thus, two beams of light co-propagate toward the distal end of the endoscope, and the reflection of the first arriving beam from the target (e.g., the object beam) interferes with the reflection of the second beam from the distal partially reflecting mirror (e.g., the second reference beam). The interference intensity pattern is collected and imaged on a camera (e.g., the detector 606).

The object beam 628, after interference from the second reference beam 619, may travel through a first lens 630, which may be the same as the first lens 130 (e.g., a concave lens). The camera system 600 further includes a second lens 632 positioned between the first lens 630 and the detector 606, with the second beam splitter 624 positioned between the first lens 630 and the second lens 632. The second beam splitter 624 may be a cube beam splitter that combines the object beam (after interference from the second reference beam) and the first reference beam 618. The second lens 632 may be the same as the second lens 132 of FIG. 1 (e.g., a convex lens).

When the object beam and the first reference beam are combined via the second beam splitter 624, their light waves intersect and interfere with each other, creating an interference pattern that is directed to the detector 606 after passing through the second lens 632. However, in some examples, the first reference beam 618 and the second beam splitter 624 may be omitted.

As explained above, the camera system 600 may be configured as an endoscope. Thus, the components described herein may be arranged in different portions of the endoscope. For example, the detector 606, the controller 640, and the ultrasound element 634 may be positioned in a first portion 602 of the endoscope. The first portion 602 may be the handle of the endoscope, and thus may include additional components not shown in FIG. 6 (e.g., a display device configured to display images generated from camera system 600). The light source 604, the prism 610, the diffuser 612, the first beam splitter 614, the neutral density filter 622, and the spatial light modulator 650 may be positioned in a second portion 603 of the endoscope. The second portion 603 may be a tube of the endoscope. The light source 604 may be operably coupled to the controller 640 via a wired connection. The first lens 630, the second beam splitter 624, the second lens 632, the partially-reflective mirror 670, and the lens system 672 may be positioned in a third portion 605 of the endoscope. The third portion 605 may be a probe of the endoscope, which may be configured to be positioned in a subject to image an internal cavity/tissue of the subject. The endoscope may include a plurality of optical fibers to enable light to travel to various components described herein. For example, the third portion 605 may include an optical fiber bundle 660. At least one optical fiber of the optical fiber bundle 660 may be an illumination fiber/bundle along which the transmission beam 616 and second reference beam 619 may travel. The remaining optical fibers of the optical fiber bundle 660 may direct the object beam 628 to the first lens 630. In other examples, the optical fiber bundle 660 may include multimode fibers configured to propagate light in both directions. The transmission beam 616 may travel from the second portion 603 to the third portion 605 (e.g., to the illumination fiber(s)) via one or more optical fibers. Likewise, the first reference beam 618 and the second reference beam 619 may travel from the second portion 603 to the third portion 605 (e.g., to the second beam splitter 624 and illumination fiber(s), respectively) via two or more respective optical fibers. The light that exits the second lens 632 may travel to the detector 606 via a plurality of optical fibers. However, in some examples, the first lens 630, the second beam splitter 624 (when included), and the second lens 632 may be positioned in the first portion 602 (e.g., the handle).

The transmission beam 616, after being modulated by the spatial light modulator 650, may impinge on a proximal side of the one or more illumination fibers included as part of the optical fiber bundle 660. In this way, the spatial light modulator 650 is illuminated with a highly coherent laser beam and/or other light sources which operates in the off-axis regime in order that modulated light is transmitted into the first order of the resulting diffraction pattern. The spatial light modulator 650 shapes the wavefront of the incident beam on the proximal end the illumination fibers which is possible to produce a diffraction-limited focus at a given distance from the distal end of the illumination fibers, so that the camera system may be used to achieve scanning-point based imaging of live samples.

Similar to the ultrasound element 134 of the camera system 100 of FIG. 1, the ultrasound element 634 may be controlled to focus the light source in the tissue, e.g., frequency shift the illumination beam at the object 626. Due to the packaging demands of the endoscope, the ultrasound element 634 may be positioned in the handle, as shown. The ultrasound waves generated by the ultrasound element 634 may travel to the object 626 via a sound guide 680 that may be packaged as part of the third portion 605/probe. However, in examples where the ultrasound element 634 may be miniaturized sufficiently to be accommodated within the third portion 605, the ultrasound element may be positioned in the third portion 605.

The controller 640 may be configured to control the light source 604 (e.g., control the pulse frequency) and the ultrasound element 634. Further, the controller 640 may be configured to receive optical information (e.g., the interference pattern) from the detector 606. The controller 640 may be configured to generate one or more images based on the optical information, or the controller 640 may be configured to send the optical information to an external computing device 642 for processing.

For example, the controller 640 may include a memory and one or more processors. The processor may control the light source, ultrasound element, and/or detector to acquire the image information described herein according to instructions stored on the memory of the controller. The processor may be in electronic communication with a display device and/or the external computing device 642, and the processor may process the image information into images for display on the display device. The processor may include a central processing unit (CPU), according to an embodiment. According to other embodiments, the processor may include other electronic components capable of carrying out processing functions, such as a digital signal processor, a field-programmable gate array (FPGA), or a graphic board. According to additional embodiments, the processor may include multiple electronic components capable of carrying out processing functions. For example, the processor may include two or more electronic components selected from a list of electronic components including: a central processor, a digital signal processor, a field-programmable gate array, and a graphic board. The memory may comprise any known data storage medium.

The lens system 672 may include a triplet Gradient Index (GRIN) lens system used for multiphoton and fluorescence endoscopes (Kim, J. K., Lee, W. M., Kim, P., Choi, M., Jung, K., Kim, S., and Yun, S. H. Fabrication and operation of GRIN probes for in vivo fluorescence cellular imaging of internal organs in small animals. Nature protocols 7(8), 1456-69 (2012), incorporated by reference herein in its entirety). The triplet-GRIN design is a modification of a single GRIN design utilizing an element with a pitch of P=1.5. GRIN lenses are fabricated in lengths relating to P=0.5. In some examples, the lens system 627 may be omitted.

The light source 604 may be configured to output light similar to the light source 104. Illumination for the camera system 600 may be, but is not limited to, 720 nm and 820 nm LED or Laser(s) described in table 1 and described in the example above. The peak wavelengths of these two sources were sufficiently separated to minimize spectral overlap so that the Bragg selectivity of each hologram only diffracts light from one source. It is useful in the differentiation between normal and diseased tissues as well as for different depths of tissues.

Raw data (e.g., from the detector 606), after complete data acquisition, are reconstructed as an image, based on a suitable algorithm, as explained above with respect to FIGS. 2-4. For example, a joint reconstruction method may be applied to avoid an error (Q. Sheng et al., “Photoacoustic computed tomography without accurate ultrasonic transducer responses,” Proc. SPIE, 9323 932313 (2015), incorporated herein by reference in its entirety). Three dimensional images may be formed. A deep neural network may be used for image reconstruction and phase recovery as well as analysis of the holographic image, as explained in Rivenson, Y., Zhang, Y., Gunaydin, H. et al. Phase recovery and holographic image reconstruction using deep learning in neural networks. Light Sci Appl 7, 17141 (2018), which is incorporated herein by reference in its entirety. Therein, using trained model non-iterative image reconstruction, twin-image suppression and phase recovery is performed.

Thus, the camera system 600 may be used to examine tissue microscopically without taking a biopsy, providing real time, easy to use, and automatic analysis of diseases and normal tissues in physicians' offices as well as in surgical operating settings.

The combination of shaping the wavefront of the incident beam on the proximal end of the optical fiber bundle by using the spatial light modulator and co-propagation of two beams toward the distal end of the endoscope by using the distal partially-reflective mirror may achieve low-coherence full-field phase-shifting holography to facilitate imaging of live samples.

The technical effect of generating a hologram based on an interference pattern generated between an object beam and a reference beam of a camera system as disclosed herein is that a high resolution (e.g., of 2-10 μm) image may be generated, and the light may have a penetration depth into diffuse medium such as the human body of more than 100-200 mm. Another technical effect of generating holograms with the camera systems as described herein is that the holograms/images may image an increased the field of view more than 16 degrees in comparison to standard modalities and uses multi-modal information and deep learning to create a hologram of the human body, allowing for three-dimensional viewing of systems and organs of interest.

The disclosure also provides support for a camera system, comprising: a light source configured to emit light in one or more wavelength ranges, a first beam splitter positioned to split the emitted light into a reference beam and a transmission beam, an aperture though which the transmission beam traverses en route to an object, and where an object beam formed from light reflected off the object is configured to travel back through the aperture, a concave lens, a convex lens, a second beam splitter positioned intermediate the concave lens and the convex lens, and a detector configured to capture an image of an interference between the reference beam and the object beam. In a first example of the system, the concave lens, the convex lens, the second beam splitter, and the detector are positioned such that the second beam splitter directs the reference beam toward the detector, the object beam is directed through the concave lens, and the reference beam and the object beam travel through the convex lens. In a second example of the system, optionally including the first example, the system further comprises: a controller configured to obtain output from the detector and generate the image based on the output. In a third example of the system, optionally including one or both of the first and second examples, the system further comprises: an ultrasound element configured to transmit and/or receive ultrasound signals. In a fourth example of the system, optionally including one or more or each of the first through third examples, the controller is configured to control the ultrasound element to transmit and receive ultrasound signals and generate an ultrasonic image based on the received ultrasound signals. In a fifth example of the system, optionally including one or more or each of the first through fourth examples, the controller is configured to control the ultrasound element to focus an ultrasonic signal to the object to wavelength-shift a portion of the transmission beam and/or the object beam. In a sixth example of the system, optionally including one or more or each of the first through fifth examples, the controller is configured to control the ultrasound element to capture photoacoustic signals generated at the object by the transmission beam.

The disclosure also provides support for a camera system, comprising: a light source configured to emit light in one or more wavelength ranges, a beam splitter positioned to split the emitted light into a reference beam and a transmission beam, a spatial light modulator positioned to modulate the transmission beam, an aperture though which the transmission beam traverses en route to an object, and where an object beam formed from light reflected off the object is configured to travel back through the aperture, a partially-reflective mirror positioned between the aperture and the object, and a detector configured to receive an interference between the reference beam and the object beam. In a first example of the system, the aperture comprises a distal end of an optical fiber bundle, and wherein the reference beam and the transmission beam travel from a proximal end of the optical fiber bundle to the distal end. In a second example of the system, optionally including the first example, the interference is created by the reference beam reflected from the partially-reflective mirror interfering with the object beam. In a third example of the system, optionally including one or both of the first and second examples, the interference is carried to the detector by the optical fiber bundle. In a fourth example of the system, optionally including one or more or each of the first through third examples, the camera system comprises an endoscope. In a fifth example of the system, optionally including one or more or each of the first through fourth examples, the system further comprises: an ultrasound element. In a sixth example of the system, optionally including one or more or each of the first through fifth examples, the system further comprises: a controller configured to control the ultrasound element and the light source such that an ultrasound wave emitted by the ultrasound element arrives at the object with the transmission beam to focus the transmission beam.

The disclosure also provides support for a method for a camera system, comprising: activating a light source of the camera system to direct a transmission beam to an object to be imaged, activating an ultrasound element of the camera system to transmit ultrasound signals to the object to be imaged, where the ultrasound signals focus the transmission beam at the object, detecting, with a detector, an interference pattern generated between an object beam and a reference beam of the camera system, the object beam comprising light from the transmission beam that has reflected off the object, and generating a hologram based on the detected interference pattern. In a first example of the method, the method further comprises: directing the object beam through a first lens and a beam splitter positioned between the first lens and a second lens, and directing the reference beam to the beam splitter lens, wherein the object beam and the reference beam are combined via the beam splitter to thereby generate the interference pattern. In a second example of the method, optionally including the first example, the method further comprises: directing the interference pattern through the second lens before the interference pattern reaches the detector, wherein the first lens is a concave lens and the second lens is a convex lens. In a third example of the method, optionally including one or both of the first and second examples, the method further comprises: modulating the transmission beam with a spatial light modulator. In a fourth example of the method, optionally including one or more or each of the first through third examples, generating the hologram based on the detected interference pattern comprises transforming the detected interference pattern to the frequency domain to generate frequency and phase domain information, filtering the frequency and phase domain information, transforming the filtered frequency and phase domain information back to the spatial domain to generate spatial domain information, extracting phase data from the spatial domain information, and generating the hologram with the phase data. In a fifth example of the method, optionally including one or more or each of the first through fourth examples, generating the hologram comprises generating an intensity-only hologram, and further comprising applying back-propagation to the intensity-only hologram to generate a phase image and an amplitude image, entering the phase image and the amplitude image as input into a model trained to perform phase recovery, and receiving, as output from the model, a recovered phase amplitude image and a recovered phase image.

References to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to a single one or multiple ones. Additionally, the words “herein,” “above,” “below” and words of similar import, when used in this application, refer to this application as a whole and not to any particular portions of this application. When the claims use the word “or” in reference to a list of two or more items, that word covers all of the following interpretations of the word: any of the items in the list, all of the items in the list and any combination of the items in the list, unless expressly limited to one or the other.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

The methods may be performed by executing stored instructions on machine readable storage media with one or more logic devices (e.g., processors) in combination with one or more additional hardware elements, such as storage devices, memory, hardware network interfaces/antennas, switches, actuators, clock circuits, etc. The described methods and associated actions may also be performed in various orders in addition to the order described in this application, in parallel, and/or simultaneously. Processors of the logic subsystem may be single core or multicore, and the programs executed thereon may be configured for parallel or distributed processing. The logic subsystem may optionally include individual components that are distributed throughout two or more devices, which may be remotely located and/or configured for coordinated processing. One or more aspects of the logic subsystem may be virtualized and executed by remotely accessible networked computing devices configured in a cloud computing configuration.

Aspects of the present disclosure are described herein with reference to flowchart illustrations and/or block diagrams of methods, apparatus, and computer program products according to the embodiments disclosed herein. Insofar as such block diagrams, flowcharts, and/or examples contain one or more functions and/or operations, it will be understood by those of skill in the art that each function and/or operation within such block diagrams, flowcharts, or examples can be implemented, individually and/or collectively, by computer readable instructions using a wide range of hardware, software, firmware, or virtually any combination thereof. The described systems are exemplary in nature and may include additional elements and/or omit elements. The subject matter of the present disclosure includes all novel and non-obvious combinations and sub-combinations of the various systems and configurations, and other features, functions, and/or properties disclosed.

As used herein, the terms “system” or “module” may include a hardware and/or software system that operates to perform one or more functions. For example, a module or system may include a computer processor, controller, or other logic-based device that performs operations based on instructions stored on a tangible and non-transitory computer readable storage medium, such as a computer memory. Alternatively, a module or system may include a hard-wired device that performs operations based on hard-wired logic of the device. Various modules or units shown in the attached figures may represent the hardware that operates based on software or hardwired instructions, the software that directs hardware to perform the operations, or a combination thereof.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

It will be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated and/or described may be performed in the sequence illustrated and/or described, in other sequences, in parallel, or omitted. Likewise, the order of the above-described processes may be changed.

The subject matter of the present disclosure includes all novel and non-obvious combinations and sub-combinations of the various processes, systems and configurations, and other features, functions, acts, and/or properties disclosed herein, as well as any and all equivalents thereof.

1. A camera system, comprising:
a light source configured to emit light in one or more wavelength ranges;
a first beam splitter positioned to split the emitted light into a reference beam and a transmission beam;
an aperture though which the transmission beam traverses en route to an object, and where an object beam formed from light reflected off the object is configured to travel back through the aperture;
a concave lens;
a convex lens;
a second beam splitter positioned intermediate the concave lens and the convex lens; and
a detector configured to capture an image of an interference between the reference beam and the object beam.

2. The camera system of claim 1, wherein the concave lens, the convex lens, the second beam splitter, and the detector are positioned such that the second beam splitter directs the reference beam toward the detector, the object beam is directed through the concave lens, and the reference beam and the object beam travel through the convex lens.

3. The camera system of claim 1, further comprising a controller configured to obtain output from the detector and generate the image based on the output.

4. The camera system of claim 3, further comprising an ultrasound element configured to transmit and/or receive ultrasound signals.

5. The camera system of claim 4, wherein the controller is configured to control the ultrasound element to transmit and receive ultrasound signals and generate an ultrasonic image based on the received ultrasound signals.

6. The camera system of claim 4, wherein the controller is configured to control the ultrasound element to focus an ultrasonic signal to the object to wavelength-shift a portion of the transmission beam and/or the object beam.

7. The camera system of claim 4, wherein the controller is configured to control the ultrasound element to capture photoacoustic signals generated at the object by the transmission beam.

8. A camera system, comprising:
a light source configured to emit light in one or more wavelength ranges;
a beam splitter positioned to split the emitted light into a reference beam and a transmission beam;
a spatial light modulator positioned to modulate the transmission beam;
an aperture though which the transmission beam traverses en route to an object, and where an object beam formed from light reflected off the object is configured to travel back through the aperture;
a partially-reflective mirror positioned between the aperture and the object; and
a detector configured to receive an interference between the reference beam and the object beam.

9. The camera system of claim 8, wherein the aperture comprises a distal end of an optical fiber bundle, and wherein the reference beam and the transmission beam travel from a proximal end of the optical fiber bundle to the distal end.

10. The camera system of claim 9, wherein the interference is created by the reference beam reflected from the partially-reflective mirror interfering with the object beam.

11. The camera system of claim 10, wherein the interference is carried to the detector by the optical fiber bundle.

12. The camera system of claim 8, wherein the camera system comprises an endoscope.

13. The camera system of claim 8, further comprising an ultrasound element.

14. The camera system of claim 13, further comprising a controller configured to control the ultrasound element and the light source such that an ultrasound wave emitted by the ultrasound element arrives at the object with the transmission beam to focus the transmission beam.

15. A method for a camera system, comprising:
activating a light source of the camera system to direct a transmission beam to an object to be imaged;
activating an ultrasound element of the camera system to transmit ultrasound signals to the object to be imaged, where the ultrasound signals focus the transmission beam at the object;
detecting, with a detector, an interference pattern generated between an object beam and a reference beam of the camera system, the object beam comprising light from the transmission beam that has reflected off the object; and
generating a hologram based on the detected interference pattern.

16. The method of claim 15, further comprising directing the object beam through a first lens and a beam splitter positioned between the first lens and a second lens, and directing the reference beam to the beam splitter, wherein the object beam and the reference beam are combined via the beam splitter to thereby generate the interference pattern.

17. The method of claim 16, further comprising directing the interference pattern through the second lens before the interference pattern reaches the detector, wherein the first lens is a concave lens and the second lens is a convex lens.

18. The method of claim 15, further comprising modulating the transmission beam with a spatial light modulator.

19. The method of claim 15, wherein generating the hologram based on the detected interference pattern comprises transforming the detected interference pattern to the frequency domain to generate frequency and phase domain information, filtering the frequency and phase domain information, transforming the filtered frequency and phase domain information back to the spatial domain to generate spatial domain information, extracting phase data from the spatial domain information, and generating the hologram with the phase data.

20. The method of claim 15, wherein generating the hologram comprises generating an intensity-only hologram, and further comprising applying back-propagation to the intensity-only hologram to generate a phase image and an amplitude image, entering the phase image and the amplitude image as input into a model trained to perform phase recovery, and receiving, as output from the model, a recovered phase amplitude image and a recovered phase image.
